(19) United States
(12) Patent Application Publication
(10) Pub. No.: US 2022/0073956 A1
(43) Pub. Date: Mar. 10, 2022

(54) SYSTEMS AND METHODS FOR RECYCLING OF REDUCED DENSITY BIOPLASTICS

(71) Applicant: Corumat, Inc., Mercer Island, WA (US)

(72) Inventors: Michael WAGGONER, Mercer Island, WA (US); Gregory J. TUDRYN, Chino, CA (US)

(73) Assignee: Corumat, Inc., Mercer Island, WA (US)

(21) Appl. No.: 17/480,672

(22) Filed: Sep. 21, 2021

Related U.S. Application Data
(63) Continuation of application No. PCT/US2020/025011, filed on Mar. 26, 2020.
(60) Provisional application No. 62/824,629, filed on Mar. 27, 2019.

(51) Int. Cl. C12P 7/56; C08J 11/10; C08J 11/14; C12P 5/02; B29B 17/02

(52) U.S. Cl. CPC C12P 7/56; C08J 11/105; C08J 11/14; C12P 5/023; B29K 2995/0056; C08J 2300/16; C08J 2367/04; B29B 2017/0244; B29B 17/02

(57) ABSTRACT

A method for deriving value from a mixed waste feedstock can include receiving a mixed waste feedstock including at least a reduced density biopolymer material and an organic feedstock. At least one of a fluid or a material that releases liquids during degradation is added to the mixed waste feedstock. The reduced density biopolymer material is separated, via density separation, from the mixed waste feedstock. The reduced density biopolymer material has a specific gravity below a specific gravity threshold. The reduced density biopolymer material separated from the mixed waste feedstock as a result of the separating is recovered.

### CROSS-REFERENCE TO RELATED APPLICATIONS

This application claims priority and benefit of U.S. Provisional Application No. 62/824,629, filed Mar. 27, 2019 and entitled “Systems and Methods for Recycling of Reduced Density Bioplastics”, the entire disclosure of which is hereby incorporated by reference herein in its entirety.

### TECHNICAL FIELD

Embodiments described herein relate generally to apparatus and methods for the recovery and re-use of biopolymers and, more particularly, to recovery of reduced density biopolymer products from multi-material amalgam via biopolymer recycling.

### BACKGROUND

Petroleum-derived plastics are widely used in both durable and nondurable (consumable) applications. One use of nondurable petroleum-derived plastics is in the packaging of goods and food service items, often in a single use application. While the use of petroleum-derived plastics can be convenient and economical, the disposal of such plastic products results in vast amounts of waste. In an effort to mitigate the issue of waste, many plastic products are made from recyclable or compostable materials. Recycling of plastics and/or other polymers has been commercial for many decades, and composting has been practiced relatively widely since the 1970's. With worldwide recovery rates below 10 percent, however, neither of these methods has achieved a desired recovery rate.

In general, recyclable polymer (e.g., plastic) products are cleaned before recycling, a process that consumes energy. Due to incomplete cleaning and degradation of the material during the process, the recovered polymer material is often unable to be recycled into the same or similar goods. Moreover, when petroleum-derived disposable plastics such as those used in food packaging are mixed with organic materials, the mixed feedstock can become contaminated and unusable in composting. Thus, while current methods of recycling and/or composting help reduce the environmental and/or health impacts associated with using and/or disposing of such materials, challenges persist.

Biopolymers and/or other alternatives to petroleum-derived polymers are intended to mitigate at least some these continuing environmental and/or health concerns. Some current biopolymers, however, are expensive relative to their petroleum-derived counterparts and/or have a limited ability to withstand the heat demands of some applications (e.g., such as some food service applications). Biopolymers and/or other cellulose fiber composites are typically designed to degrade in composting environments and therefore, are not usually recycled. During composting, these compostable materials are generally mixed in with other organic and compostable materials such as food wastes, packaging, paper materials, and low-value organics, and then digested to produce biogas (methane), humus (a good soil additive), and/or the like. While the output from digestion is typically considered useful, it results in relatively low value products, especially when compared to the value of recycled plastics.

Some known methods of recycling biopolymers have included, for example, compaction, separation, or solvation followed by recovery of the biopolymer, which may ultimately disrupt the original structure and morphology of the biopolymer. Moreover, such methods of recycling may consume large amounts of energy and result in relatively high cost for purification. As such, recycling and/or composting biopolymers provides limited economic incentive for the recovery and re-use of such materials.

Accordingly, a need exists for systems and methods for recycling of biopolymer materials such as, for example, reduced density biopolymers and/or other compostable alternatives to petroleum-derived polymers.

### SUMMARY

Systems and methods to recover reduced density bioplastic products from multi-material amalgam via biopolymer recycling are described herein. In some embodiments, a method for deriving value from a mixed waste feedstock can include receiving a mixed waste feedstock including at least a reduced density biopolymer material and an organic feedstock. At least one of a fluid or a material that releases liquids during degradation is added to the mixed waste feedstock. The reduced density biopolymer material with a specific gravity below a specific gravity threshold is separated, via density separation, from the mixed waste feedstock. The method includes recovering the reduced density biopolymer material separated from the mixed waste feedstock as a result of the density separation.

### BRIEF DESCRIPTION OF THE DRAWINGS

FIG. 1 is a flowchart illustrating a process of recycling a biopolymer material, according to an embodiment.

### DETAILED DESCRIPTION

The systems and methods described herein are generally directed to facilities, materials, practices, and/or methods that may be used to recover and/or recycle materials such as polymers and/or biopolymers. While specific examples of recycling processes and/or methods are described herein, it should be understood that they have been provided by way of example only and not limitation. For example, in some instances, the systems and/or methods described herein can be used to recycle, recover, concentrate, repurpose, and then reuse biopolymers and/or biopolymer constituents such as oligomers, monomers, and/or the like derived from recycled biopolymers. In some instances, the systems and methods can create and/or increase value by recovering useful biopolymers and/or bio-based oligomers and monomers from a mixed waste feedstock. The recovery of the biopolymers and/or bio-based oligomers and monomers can be achieved via any suitable combination of recycling and composting processes and/or steps. In addition, any suitable combination of recycling and composting processes and/or steps can produce and/or can result in production of a biogas, which can be provided to heating and/or drying equipment, or turbines to generate electricity. In some implementations, the generated electricity may be sufficient to power at least a portion of the equipment used in the recycling process, an entire facility, and/or may be sold and/or otherwise delivered to the grid or any suitable energy storage facility.

In some embodiments, a method for deriving value from a mixed waste feedstock can include receiving a mixed waste feedstock including at least a reduced density biopolymer material and an organic feedstock. At least one of a fluid or a material that releases liquids during degradation is added to the mixed waste feedstock. The reduced density biopolymer material is separated, via density separation, from the mixed waste feedstock. The reduced density biopolymer material has a specific gravity below a specific gravity threshold. The reduced density biopolymer material separated from the mixed waste feedstock as a result of the separating is recovered. In some embodiments, the method can further include optionally fermenting a portion of the mixed waste feedstock including material that has a specific gravity greater than the specific gravity threshold. Optionally, biogas can be generated as a result of the fermenting and/or a biopolymer material can be generated from the fermented mixed waste feedstock via an organism.

As used in this specification, the singular forms “a,” “an,” and “the” include plural referents unless the context clearly dictates otherwise. Thus, for example, the term “a member” is intended to mean a single member or a combination of members, “a material” is intended to mean one or more materials, or a combination thereof.

As used herein, a “biopolymer” refers generally to a polymer (a large molecule composed of many repeating units known as monomers) that is produced by or from a living organism. Biopolymers produced from a living organism generally are produced by chemical processing the organism (e.g., plant) into a chemical that serves as the monomer for the biopolymer. Biopolymers can be biodegradable or non-biodegradable. Non-biodegradable biopolymers, for example, can form polymers that are substantially the same as the corresponding petroleum-derived polymers. Biodegradable biopolymers refer to a polymer that is produced by or from a living organism and that is configured to be consumed by bacteria, degrade in organic waste or compost, and/or naturally degrade over a given time. Non-limiting examples of biodegradable biopolymers can include polylactic acid (PLA), polybutylene succinate (PBS), polyhydroxyalkanoate (PHA), polyhydroxlybutyrate (PHB), poly(3-hydroxybutyrate-co-hydroxyhexanoate) (PHBH), cellulose, starch, and/or the like. In some instances, a biopolymer product can be formed from and/or can include one or more biopolymer materials and/or a composite of multiple biopolymer materials (e.g., either biodegradable, non-biodegradable, or a combination thereof). While the embodiments and/or examples provided herein are described as being used to recover and/or otherwise recycle biodegradable biopolymers, it should be understood that the embodiments and/or examples can be used to recover and/or otherwise recycle biodegradable biopolymers, non-biodegradable biopolymers, and/or non-biopolymers. In some embodiments, the non-biopolymer may be polybutylene succinate (PBS), biodegradable aliphatic polyester, or PLA produced by a catalytic process (ethyl lactate extraction) rather than fermented plant starch. More particularly, while some of the embodiments and/or examples provided herein are described as being used to recover and/or otherwise recycle PLA, it should be understood that the embodiments and/or examples provided herein can be used to recycle and/or recover any suitable biodegradable biopolymer. In other words, any of the systems and/or methods described herein may be used with and/or maybe be modified for use with any suitable biopolymer, bio-based material, non-biopolymer, and/or non-bio-based material.

Polylactic acids also referred to as polylactides or PLA are a lactic acid-based biopolymer. PLA is a biodegradable aliphatic polyester that has a wide range of applications in various fields and the raw material—lactic acid (LA) or 2-hydroxypropionic acid—can be derived from renewable resources. For example, PLA can be produced either by ring-opening polymerization (ROP) of lactides or by condensation polymerization of the lactic acid monomers, which can be obtained from the fermentation of corn, beet-sugar, cane-sugar, etc.

In general, PLA has high mechanical properties, thermal plasticity, fabricability, and biocompatibility, and can readily degrade in commercial composting environments. In some instances, these properties and/or characteristics can make PLA a suitable and/or preferred material for use in sustainable packaging. Moreover, PLA can be expanded and/or foamed to reduce a density of the constituent materials, which in turn, can be used to form low density or reduced density products (e.g., low density or reduced density packaging). In some instances, the low density or reduced density packaging can have increased flexibility, resilience, and/or heat tolerance that can be suitable for use in food packaging. For example, expanded and/or foamed PLA can be used in food packaging including takeout food containers, clamshell food packaging containers, meat trays, bowls, cups, lids, cutlery, and agricultural packaging such as berry containers, seed starters, fruit trays, egg cartons, mushroom tills among any number of other applications. Because PLA is a biodegradable biopolymer, PLA food packaging products and more particularly, expanded PLA food packaging products, can be disposed of alongside or in contact with waste organics without contaminating a compost or organic waste feedstock.

The systems and/or methods described herein can be used to recover reduced density biopolymer products (e.g., expanded PLA products) that have a density below a density threshold. For example, in general, PLA can have a density of approximately 1.2 grams per cubic centimeter (g/cc) and expanded and/or foamed PLA can have a reduced density between about 0.06 g/cc to about 0.2 g/cc. In some instances, expanded and/or foamed PLA can have a specific gravity (e.g., a value representing a ratio between a density of the biopolymer and a density of water) below, for example, 0.9, which can enable the expanded and/or foamed PLA to float when in a volume of water or other fluids, a slurry, and/or the like. As described in further detail herein, the systems and/or methods described herein can use density separation and/or other suitable techniques to recover reduced density PLA and/or other biopolymer products.

Expanded and/or foamed polymers (e.g., petroleum-based polymers and/or biopolymers) can be formed by introducing a blowing agent or an inert gas into a polymeric resin that is then allowed to cure into the expanded and/or foamed polymer. In some instances, systems and/or methods for expanding PLA to form low-density products can be similar to and/or substantially the same as any of those described in, for example, U.S. Pat. No. 10,513,590 entitled, “Reduced Density Thermoplastics,” filed Jun. 19, 2014 (referred to herein as “the '590 patent”); and/or U.S. Patent Publication No. 2016/0121577 entitled. “Layered Structures,” filed Nov. 2, 2015 (referred to herein as “the '577 publication”), the disclosure of each of which is incorporated herein by reference in its entirety. For example, expanded and/or foamed polymeric articles can be made from multi-layer cellular structures with or without an outer skin, as described in detail in the '577 publication. The multi-layer cellular structures can include, for example, non-laminated multi-layer polymer sheets forming two discrete outer layers that enclose, bound, and/or sandwich any number of inner foamed layers having one or more discrete cell sizes. In some instances, an expanded and/or foamed polymer article formed of a number of layered structures of expanded cells with at least two distinct cell sizes (e.g., up to approximately 50 micrometers (μm) in diameter for smaller cells and greater than approximately 51 μm in diameter for larger cells) may provide weight reduction, while enabling the expanded and/or foamed polymer to retain desirable mechanical performance during use.

In other instances, reduced density polymers (biopolymers) can be expanded, foamed, and/or layered via any suitable technique and/or method. For example, a reduced density biopolymer can be formed to include a layered structure with relatively hard outer layers, which may enable the reduced density biopolymer to be used in a wide range of durable applications and/or in applications for which conventionally foamed polymers may not have been suitable. In some instances, such a layered biopolymer can have a hardness or durometer of 35 Shore A or greater. In other instances, a layered biopolymer can have a durometer that is less than 35 Shore A. In still other instances, the systems and/or methods described herein can be used to recover and/or recycle a reduced density biopolymer that was formed via conventional foaming methods. For example, in some instances, reduced density PLA can be formed via lamination and can include any number of layered structures that can be foamed via extrusion foaming and/or the like. In some instances, a reduced density PLA product used, for example, in food packaging and/or the like, can include a dissolvable or otherwise water-soluble barrier layer configured to limit and/or substantially prevent saturation of the reduced density PLA product. In such instances, the reduced density PLA product can be such that the dissolvable and/or water-soluble barrier layer is released, degraded, and/or digested during recovery using the systems and/or methods described herein. In other instances, reduced density PLA material or product can include chain extenders and/or can have chain lengths of over 130,000, which may be separated during recovery (using the systems and/or methods described herein) due to differences in viscosity and/or different responses to reactions such as, for example, hydrolysis.

As described below with reference to specific examples, the expanded and/or foamed polymers recovered and/or recycled using the systems and methods described herein can be, for example, semi-crystalline biopolymers such as PLA, PHA, starch, cellulose, and/or the like, or blends thereof. The resulting expanded and/or foamed biopolymer article can be suitable for use in food packaging (and/or other applications) based at least in part on the biopolymer being compostable. In addition, the reduced density of the expanded and/or foamed biopolymer can enable the material to float on water and/or on other relatively low-density fluids or slurries, which in turn, can allow for recovery of the expanded and/or foamed biopolymer via one or more density separation processes (e.g., float separation). More specifically, the systems and/or methods described herein can enable expanded and/or foamed biopolymers (e.g., PLA or the like) to be incorporated and/or otherwise included in an organic waste stream and can be configured to separate and/or recover at least a portion of the expanded and/or foamed biopolymer, via density separation (e.g., float separation), either prior to or during fermentation, anaerobic digestion, and/or other modes of composting the organic waste stream. Furthermore, the systems and/or methods described herein can be performed such that an output of the fermentation, anaerobic digestion, and/or other modes of composting the organic waste stream is at least one of lactic acid, which can then be polymerized to form PLA, one or more oligomers used for nucleating longer chains during polymerization, and/or a useable biogas (e.g., methane), which can then be used to power one or more electric turbines. In some embodiments, the expanded and/or foamed polymers recovered and/or recycled using the systems and methods described herein can be, for example, petroleum-based polymers such as polyethylene terephthalate.

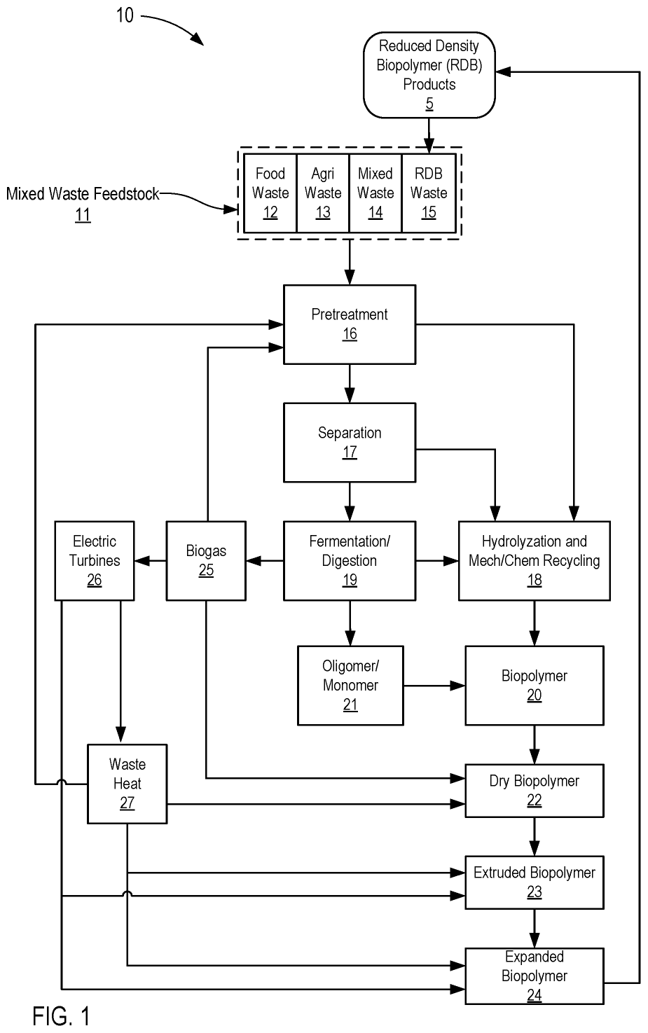

Examples of recovering and/or recycling a biopolymer such as PLA are provided below with reference to FIG. 1. While particular systems, devices, and/or methods are described below it should be understood that they have been presented by way of example only and not limitation. Accordingly, other systems, devices, and/or methods that function to recover and/or recycle a biopolymer are contemplated. While events, processes, and/or steps are described in the examples below as being performed in a particular order, it should be understood that the order of such events, processes, and/or steps may be modified or changed. Moreover, any of the events, processes, and/or steps may be performed concurrently or at least partially concurrently in a combined process or in one or more parallel processes.

FIG. 1 is a flowchart illustrating a process flow through a system 10 for recovering and/or recycling products formed of or from one or more biopolymers. More particularly, as described above, the biopolymer products can be reduced density biopolymer (RDB) products 5 such as, for example, products formed of or from expanded and/or foamed PLA. In some instances, at least a portion of the reduced density biopolymer materials forming the RDB products 5 can have a known and/or predetermined density that is less than a density threshold. In some instances, the density threshold can be at least partially based on a density of water (e.g., at or substantially at sea level). For example, the reduced density biopolymer materials forming the RDB products 5 can have a density that is less than a density of water. Said another way, the reduced density biopolymer materials forming the RDB products 5 can have a specific gravity that is less than, for example, 1.0 (e.g., a specific gravity threshold). In some embodiments, specific gravity threshold can be equal to about 0.9. In other embodiments, a specific gravity threshold can be less than 0.9 or can be greater than 0.9. As described in further detail herein, the system 10 can be configured to separate materials (e.g., biopolymers such as reduced density PLA) in a waste stream and/or feedstock that have a density below a density threshold and/or a specific gravity below a specific gravity threshold from other materials in the waste stream and/or feedstock that have a density above the density threshold and/or a specific gravity above the specific gravity threshold.

The RDB products 5 can be used (e.g., by a consumer) and can be discarded as RDB waste 15. In some instances, the RDB products 5 can be disposable and/or single use food service products formed of or from, for example, reduced density PLA. In other instances, the RDB products 5 can be formed of or from any suitable biopolymer and used in any suitable manner. As shown in FIG. 1, the system 10 can enable the RDB waste 15 to be mixed into and/or otherwise included in a mixed waste feedstock 11 that is received at a recycling facility and/or the like. In some instances, the mixed waste feedstock 11 can be an organic feedstock that can include, for example, food waste 12, agricultural waste 13, mixed waste 14, and the RDB waste 15 (e.g., reduced density PLA). In some instances, the mixed waste feedstock 11 can be received from a source that limits the use of non-biopolymers having a specific gravity that is below the specific gravity threshold (e.g., a specific gravity below 0.9). In other instances, the mixed waste feedstock 11 can be received from a source that limits the use of any or all non-biopolymers. In some instances, the mixed waste feedstock 11 can be received from a location and/or a source where at least 10% of food service items are made from reduced density biopolymers such as, for example, the reduced density PLA.

In some instances, the mixed waste feedstock 11 can be source separated to remove undesirable materials from the mixed waste feedstock 11. For example, in some instances. source separation can include a grouping or separating of organic waste material from inorganic waste material. In such instances, the source separation can limit an amount of non-biopolymer material, metal material, and/or the like that is included in the mixed waste feedstock 11. In some instances, source separation can include grouping recyclable material and compostable material into a first group, and grouping non-recyclable and non-compostable material into a second group that is separated from the first group.

The system 10 can be configured such that the facility or a portion of the facility receives the mixed waste feedstock 11 that has been pre-mixed and/or otherwise comingled prior to reaching the facility (e.g., at the waste source and/or during source separation). In other instances, the system 10 can be configured such that the facility or a portion of the facility receives separate waste streams including, for example, a waste stream of the food waste 12, a waste stream of the agricultural waste 13, a waste stream of the mixed waste 14, and a waste stream of the RDB waste PLA 15, which are then combined at the facility to collectively form the mixed waste feedstock 11. In some instances, the food waste 12, the agricultural waste 13, and the mixed waste 14 can be combined prior to reaching the facility and/or combined at the facility prior adding the RDB waste 15 to the mixed waste feedstock 11. For example, in some instances, the RDB waste 15 can be received at the facility independent of other waste streams. In some instances, it may be desirable to financially incentivize the delivery of RDB waste PLA 15 to the facility (e.g., providing payment for a given quantity reduced density PLA received).

As shown in FIG. 1, after receiving the mixed waste feedstock 11, the system 10 can be configured to perform one or more processes associated with pretreatment 16. In some instances, the pretreatment 16 can include, for example, any suitable shredding process. separating process, sterilization process, and/or the like. For example, in some embodiments, the mixed waste feedstock 11 can be conveyed into a shredder configured to reduce the size of the constituents of the mixed waste feedstock 11 to a suitable size. In some instances, a shredder can be configured to reduce a size of the constituent material to a size between 1.0 inch (in.) and about 12.0 in., between about 2.0 in. and about 11.0 in., between about 3.0 in. and about 10.0 in., between about 4.0 in. and about 9.0 in. between about 5.0 in. and about 8.0 in., or between about 6.0 in. and about 7.0 in or any suitable size or range of sizes therebetween. In other instances, the shredder can reduce the size of the constituent materials to a size that is less than 1.0 in. or that is greater than 12.0 in. In some instances, the shredder can be configured to reduce the size of the constituent materials in the mixed waste feedstock 11 to a size that is suitable and/or desirable for one or more subsequent separation processes. In addition, some very low density materials (e.g., with a specific gravity below 0.06) may not be suitable for shredding, which can enable such materials to be removed from the mixed waste feedstock 11.

In some embodiments, the pretreatment 16 can include separating from the mixed waste feedstock 11 constituent material with a size greater than a threshold size prior to shredding (e.g., between about 0.375 in. to about 1.0 in., or any other suitable size). For example, the mixed waste feedstock 11 can be conveyed to or along one or more screens. grates, filters, skimmers, rakes, and/or the like configured to remove constituent materials (e.g., biopolymer material or any other material) having a size above the threshold size. In some embodiments, such separation can be performed, at least in part, via visual inspection and manual removal (e.g., by a human) of relatively large pieces of the RDB waste 15 prior to shredding. In some instances, any amount of the RDB waste 15 that is separated can be recovered and provided to one or more subsequent processes, as described in further detail herein. Conversely, relatively large pieces of non-recyclable material, non-compostable material, inorganic material, and/or any other undesirable material can be removed from the mixed waste feedstock 11 to limit an amount of undesirable material provided to one or more subsequent processes. In other instances, the pretreatment 16 need not include such size separation.

In some embodiments, the pretreatment 16 can include separating and/or sorting the mixed waste feedstock 11, for example, based on density, relative density, and/or specific gravity. For example, in some embodiments, the system 10 can convey the mixed waste feedstock 11 to or through one or more centrifugal separators and/or centripetal force separators configured to sort and/or otherwise arrange the mixed waste feedstock 11 in a desired density gradient and/or the like. In some embodiments, the system 10 can convey the mixed waste feedstock 11 to or through one or more fluid bed separators and/or cyclonic separators having a flow rate configured to entrain, separate, and/or remove constituent materials having a specific gravity or a relative density (e.g., a ratio of the density of the material relative to, for example, a fluid in a fluid bed separator and/or the like) below a specific gravity or a relative density threshold. In some instances, any amount of the RDB waste 15 that is separated can be recovered and provided to one or more subsequent processes, as described in further detail herein.

While it may be desirable for the mixed waste feedstock 11 to include only the food waste 12, the agricultural waste 13, the mixed waste 14, and the RDB waste 15 (e.g., reduced density PLA), in some instances, the mixed waste feedstock 11 may include undesirable constituent materials. Accordingly, the pretreatment 16 can include separating and/or removing the undesirable constituent materials from the mixed waste feedstock 11. For example, in some embodiments, the system 10 can convey the mixed waste feedstock 11 to or through a magnetic separator configured to remove magnetic materials such as ferrous metals. In some embodiments, the system 10 can convey the mixed waste feedstock 11 to or through an eddy current separator configured to remove nonferrous metals. In some embodiments, the system 10 can convey the mixed waste feedstock 11 to or through one or more glass separators. optical separators, disc separators, and/or the like to remove any other undesirable material. Moreover, any such separation or the like can be performed before, during, or after shredding the mixed waste feedstock 11. As such, the system 10 can be configured to use any suitable processes and/or steps to limit undesirable materials in the mixed waste feedstock 11.

In some embodiments, the pretreatment 16 can include sterilizing the mixed waste feedstock 11. For example, in some embodiments, the system 10 can convey the mixed waste feedstock 11 into a chamber of an autoclave tumbler or the like. In such embodiments, after receiving the mixed waste feedstock 11, a volume of air (e.g., substantially all the air) in the chamber can be evacuated and a volume of hot pressurized steam can be conveyed into the chamber. The evacuation of the air and the filling of the chamber with pressurized steam can be performed sequentially or in substantially parallel processes. In some embodiments, a sterilization function as described with reference to the autoclave tumbler, can be included in and/or otherwise integrated into any other suitable device such as, for example, a shredder, a pyrolizer, a separator, a fermenter, a digester, and/or the like.

In some embodiments, after the pretreatment 16, the system 10 can convey the mixed waste feedstock 11 to or through one or more separators (represented in FIG. 1 as “Separation 17”) configured to separate constituent materials of the mixed waste feedstock 11. For example, in some instances, the separation 17 can include float separation in which a desired specific gravity threshold can be defined, set, and/or predetermined such that constituent materials having a specific gravity below the threshold (e.g., the RDB waste 15 and/or any other waste materials) float on a surface of a fluid in the float separator and constituent materials having a specific gravity above the threshold do not float (e.g., such materials can sink or substantially sink in the fluid). In some instances, the specific gravity threshold can be, for example, about 0.9. In other instances, the specific gravity threshold can be less than 0.9 or greater than 0.9. In some embodiments, the system 10 can convey the mixed waste feedstock 11 into a chamber, volume, tank, vat, etc. and can add a volume of a fluid, liquid, and/or slurry into the chamber, volume, tank, vat, etc. Moreover, in some instances, the fluid, liquid, and/or slurry can be selected based on and/or can otherwise have a desired density at least partially associated with the specific gravity threshold. As such, constituent material having a specific gravity below the specific gravity threshold (e.g., at least a portion of the RDB waste 15) can float on a surface of the fluid (e.g., water), liquid, and/or slurry and can be recovered via a skimmer, rake, screen, and/or any other recovering device or mechanism.

While the separation 17 is described above as including and/or performing float separation, in other embodiments, the separation 17 can include and/or perform separation of any suitable kind. In some embodiments, for example, the separation 17 can include density separation via one or more methods in addition to or instead of float separation. In other embodiments, the separation 17 can include other modes of separation as in addition to or instead of density separation such as, for example, any of those described above with reference to the pretreatment 16. While the separation 17 is described above as being performed after the pretreatment 16, in other embodiments, the separation 17 can be included in the pretreatment 16 (e.g., as a final step) or can be performed in combination with one or more subsequent processes, as described in further detail herein.

In some instances, limiting and/or pre-separating an amount of constituent materials in and/or from the mixed waste stream 11 that are not bio-based and/or organic waste can allow for recovery of constituent material that is substantially all reduced density biopolymers during the separation 17 (e.g., density separation and more specifically, float separation). In other instances, non-biopolymers, biopolymers with coatings, barrier layers, and/or impurities (e.g., materials that are not the specific biopolymer) can be included in the mixed waste feedstock during the separation 17. In some such instances, the separation 17 can include float separating the mixed waste feedstock 11 and a surfactant and/or any other suitable additive, enzyme. and/or the like can be conveyed into the volume of fluid during the float separation to facilitate and/or enhance separation of hydrophobic materials from hydrophilic materials and/or to increase or decrease an amount of adsorption between the constituent materials in the mixed waste feedstock 11 and the fluid, liquid, and/or slurry. The surfactant (or other additive) can be any suitable material, chemical, formula, etc. In some embodiments, a surfactant can be and/or can include a detergent or solvent configured to wash particulates from a constituent material that would otherwise have a specific gravity below the specific gravity threshold. For example, in some instances, a detergent or solvent may be operable to remove from a piece of reduced density PLA food residue that may prevent the reduced density PLA from floating during the float separation (or any other mode of separation performed during the separation 17). In some instances, air can be introduced into the fluid, liquid, and/or slurry to facilitate separation during the float separation. For example, the air can facilitate separation of constituent materials by reducing an effective density of the fluid, liquid, and/or slurry. As such, constituent materials that otherwise have a density close to that of the fluid, liquid, and/or slurry (e.g., a relative density and/or a specific gravity close to or equal to 1.0) can sink while, for example, the RDB waste 15 continue to float on a surface of the fluid, liquid, and/or slurry.

Constituent materials that are separated and recovered during the pretreatment 16 and/or the separation 17 (referred to herein as “separated materials”) can be recycled via any suitable process. As shown in FIG. 1, the system 10 can be configured to process the separated materials (e.g., materials such as the RDB waste 15 and/or any other material having a specific gravity below the specific gravity threshold) via hydrolyzation and/or mechanical or chemical (“Mech/Chem”) recycling 18 (also referred to herein as “the recycling 18”). More particularly. in some instances, the system 10 can be configured to process via the recycling 18 relatively high quality RDB waste 15 that was recovered via the pretreatment 16 and/or the separation 17 (e.g., float separation). Said another way, the system 10 can be configured to limit an amount of non-biopolymer materials in the waste stream conveyed to the recycling 18. In some instances, the limiting of the non-biopolymer material can be a function of the pretreatment 16 and/or the separation 17. In other instances, the system 10 can perform one or more processes to remove recovered, non-biopolymer materials included in the recovered waste stream. In some instances, recycling 18 the high quality RDB waste 15 can result in a higher efficiency of the recycling 18 and/or can result in a higher quality output of the recycling 18.

The hydrolyzation and/or mechanical or chemical recycling 18 can include any number of processes intended to break down the separated materials into the constituent components of that material. For example, in some embodiments, mechanical recycling can include further separation of the separated materials such that each material is processed individually. Mechanical recycling can further include using mechanical means to wash, shred, granulate, degrade, and/or otherwise breakdown the material and melting down the material into a resin that can be used to create new products. In some instances, the RDB waste 15 can include, for example, reduced density PLA that has a barrier layer or the like (as described above), which can be separated, dissolved, and/or otherwise removed from the reduced density PLA during one or more mechanical recycling processes.

In some embodiments, chemical recycling can include any suitable thermochemical reaction configured to reduce a molecular weight of a material and/or to breakdown the material into its chemical components or building blocks such as monomers and/or oligomers. For example, chemical recycling of a biopolymer can include, for example, dissolving the biopolymer in a solvent and heating the mixture to a desired temperature to produce, generate, and/or otherwise form monomers and/or oligomers associated with that biopolymer and/or a form of the biopolymer having a reduced molecular weight. Moreover, the monomers, oligomers, and/or the reduced molecular weight biopolymer can then be polymerized (or repolymerized) to form or reform that biopolymer (represented in FIG. 1 as “Biopolymer 20”). In some instances, chemical recycling of reduced density PLA can include, for example, performing a degradation process or reaction (e.g., a hydrolysis process or reaction, and/or the like). For example, in some instances, a hydrolysis process or reaction can be performed in which a water molecule is consumed to separate the PLA molecule into its chemical components. More specifically, the hydrolyzation of the reduced density PLA can result in oligomeric PLA and/or the formation of lactic acid monomers or lactide, which can then be polymerized to form PLA. The PLA (e.g., the biopolymer 20) can be, for example, high density and/or high molecular weight PLA. Said another way, the biopolymer 20 (e.g., the PLA and/or any other biopolymer) is a non-expanded and/or a non-foamed biopolymer.

By way of example, in some embodiments, the recycling 18 can include hydrolyzation of PLA (e.g., reduced density PLA and/or non-reduced density PLA). The hydrolyzation can include, for example, dissolving the PLA in response to being exposed to one or more of water, alcohol, glycerin, glycol, sodium hydroxide, propanol, butanol, methyl acetate, trimethylamine, methyl ethyl ketone, and/or any other solvents, enzymes, and/or the like. The solution can then be heated, for example, to a temperature of about 80° C. at atmospheric or at substantially atmospheric pressures. In other instances, hydrolyzation can be performed at a temperature of about 95° C., at about atmospheric pressure, and for a time of about 12-24 hrs. In other instances, hydrolyzation can be performed at a temperature of about 130° C., at a pressure of at least 20.0 pounds per square inch gauge (psig), and for a time of about 2 hrs. In still other instances, hydrolyzation can be performed at any suitable temperature (e.g., less than 80° C. or greater than 80° C.), at a pressure less than atmospheric pressure or greater than atmospheric pressure, and/or for any suitable time. In some instance, the hydrolysis reaction of PLA can be different than a reaction of other biopolymers and/or non-biopolymers and thus, the hydrolyzation can allow for further separation and/or purification of the separated materials (e.g., polystyrene and/or PET, for example, are not broken down during hydrolysis of PLA).

In some instances, the PLA can include, for example, a barrier layer (as described above), which can be separated, dissolved, solubilized, and/or otherwise removed from the PLA during one or more chemical recycling processes. For example, in some instances, the hydrolyzation of the PLA can be operable to separate the barrier layer from the PLA. In other embodiments, the barrier layer can be solubilized prior to, during, and/or after the hydrolyzation of the PLA. In some instances, removing the barrier layer from the PLA substrate or material can, for example, increase a purity of the lactide oligomers and/or monomers resulting from the recycling 18.

As shown in FIG. 1, the system 10 can be configured to process the mixed waste feedstock 11 (e.g., at least a portion of the mixed waste feedstock 11 not previously separated and/or recovered) via fermentation and/or digestion 19. In some embodiments, the system 10 can be configured to convey the mixed waste feedstock 11 into a fermenter and/or a digester (e.g., an aerobic digester or an anaerobic digester). In addition, the system 10 can be configured to convey a fluid, a liquid, a slurry, a culture medium, and/or any suitable organic material that releases liquid during degradation. In some embodiments, the fermentation and/or digestion 19 can include, for example, one or more sterilization processes after the mixed waste feedstock 11 is conveyed into the fermenter and/or digester. In such embodiments, the sterilization can be substantially similar to the sterilization described above with reference to the pretreatment 16 and can be performed, for example, prior to fermentation and/or digestion.

In some embodiments, the fermenter and/or digester can further be configured to perform separation (e.g., float separation and/or the like) prior to, during, and/or after one or more fermentation and/or digestion processes. In such embodiments, the separation can be substantially similar to the separation 17 described above. In some embodiments, float separation can be performed in the fermenter and/or digester and can be based on and/or can use a specific gravity threshold that is less than the specific gravity threshold described above with reference to the float separation (e.g., the separation 17), substantially equal to the specific gravity threshold described above with reference to the float separation, or greater than the specific gravity threshold described above with reference to the float separation. In some embodiments, the float separation can be performed in the fermenter and/or digester prior to, during, and/or after fermentation and/or digestion. Said another way, the separation 17 (e.g., float separation) and the fermentation and/or digestion 19 can be combined into a single process and/or can be multiple processes performed in or using the same equipment.

Accordingly, in some embodiments, the system 10 can be configured to recover constituent materials with a specific gravity below a specific gravity threshold and/or that are otherwise floating on a surface of a fluid, liquid, and/or slurry included in the fermenter and/or digester, as described in detail above. In some instances, a surfactant can be added to the mixture and/or a volume of air or gas can be injected into the fluid, liquid, and/or slurry contained in the fermenter and/or digester. In some instances, steam injected into the fermenter and/or digester can further facilitate and/or enhance the float separation and/or any other suitable density separation. In some embodiments, the system 10 can be configured to recover the separated material (e.g., the RDB waste 15 such as reduced density PLA) and to convey the separated material into the waste stream processed via the recycling 18. In other embodiments, the fermentation and/or digestion 19 need not include the separation 17.

The fermentation and/or digestion 19 can include any suitable fermentation and/or digestion process configured to ferment and/or digest organic waste material in the mixed waste feedstock 11 into desirable chemical compounds such as monomers and/or oligomers. More specifically, in the example shown in FIG. 1, the constituent material having a specific gravity greater than the specific gravity threshold and/or otherwise not previously separated or recovered (e.g., the organic waste material) can be fermented and/or digested to generate, produce, and/or otherwise result in reduced molecular weight form of the biopolymer and/or one or more desired monomers, oligomers, and/or the like based on the desired biopolymer to be produced (represented in FIG. 1 as “Oligomer/Monomer 21”).

By way of example, in some instances, the desired biopolymer to be produced is PLA. In such instances, the fermentation and/or digestion 19 can be configured to generate lactic acid monomers and/or lactide. More specifically, the system 10 can be configured to use any suitable culture or microorganism and/or any suitable process to ferment and/or digest the organic waste material to generate the lactic acid monomers and/or lactide. For example, in some embodiments, the organic waste material can be fermented and/or digested using a culture of Lactobacillus rhamnosus (ATCC 10863). In some instances, using the Lactobacillus rhamnosus (L. rhamnosus) microorganism can include saccharification of the carbon source prior to fermentation. After saccharification, the inoculated culture can be added and/or conveyed into the fermenter and/or digester. In some instances, it may be desirable to maintain a pH of the mixture contained in the fermenter and/or digester between about 5.5 and about 6.0. In some instances, it may be desirable to add a sodium hydroxide (NaOH) to the mixture contained in the fermenter and/or digester to control and/or modulate the pH of the mixture. In some instances, fermentation and/or digestion can be when the pH of the mixture substantially equalizes and/or rate of delivering NaOH into the mixture falls below a predetermined threshold rate. Accordingly, the fermentation of the constituent material having a specific gravity above the specific gravity threshold can generate, produce, yield, and/or otherwise result in a quantity of lactic acid.

As shown in FIG. 1, the system 10 can be configured to repolymerize oligomers/monomers 21 generated during the fermentation and/or digestion into the desired biopolymer 20. For example, in some instances, lactic acid generated during the fermentation can be repolymerized (or used as a nucleating agent) in the same process as or in a process parallel to the repolymerization of the lactic acid produced during the recycling 18 (e.g., hydrolysis), thereby generating the PLA. More specifically, in some instances, the repolymerization of the lactic acid into PLA can include, for example, dehydrating the lactic acid and/or a solution including the lactic acid. The dehydrated lactic acid or lactic acid solution can then be used to form lactic acid (lactide) oligomers and/or lactide monomers. In other instances, the dehydrated lactic acid can be further processed and/or otherwise used to form any other suitable oligomers or monomers associated with, for example, any suitable polymer and/or biopolymer. In some instances, the lactide oligomers and/or lactide monomers can be polymerized into a resin using one or more catalysts (e.g., a metal catalyst) and thermal energy (e.g., heat). As described above, the resulting PLA can be, for example, high density and/or high molecular weight PLA (i.e., non-expanded and/or non-foamed PLA).

While the system 10 is described above as generating lactic acid during the recycling 18 and/or the fermentation and/or digestion 19, which in turn, is repolymerized into PLA, in some instances, the system 10 can be configured to use the recovered reduced density PLA as a nucleator or an initiator in the production of one or more other biopolymers. For example, in some embodiments, at least a portion of the recovered reduced density PLA can be used to form a nucleus and/or otherwise used to initiate a polymerization of one or more biopolymers (e.g., a biopolymer other than PLA).

In some instances, after the repolymerization of the oligomers and/or monomers into the biopolymer 20 resin (e.g., the lactide oligomers and/or monomers into the PLA resin), the system 10 can be configured to dry the biopolymer (represented in FIG. 1 as “Dry Biopolymer 22”). In some instances, the dried, higher density or non-expanded biopolymer (e.g., PLA) can be used to generate higher density or non-expanded biopolymer products. As such, the RDB products 5 can be recovered, recycled, and manufactured into non-reduced density or higher density biopolymer products. Moreover, in some instances, these biopolymer products can be used and discarded. In turn, the biopolymer waste can be included in the mixed waste feedstock 11, as described in detail above.

In other instances, the system 10 can be configured to recover and recycle the RDB products 5 into the same or different reduced density biopolymer products 15. For example, after drying the biopolymer (represented in FIG. 1 as “Dry Biopolymer 22”), the system 10 can be configured to extrude the biopolymer into one or more sheets and/or the like (represented in FIG. 1 as “Extruded Biopolymer 23”). The system 10 can then be configured to expand the biopolymer and/or otherwise reduce the density of the biopolymer using any of the processes and/or methods described herein (represented in FIG. 1 as “Expanded Biopolymer 24”). As such, the RDB products 5 can be received by the system 10 as RDB waste 15, which can be recovered, recycled, and manufactured into the same or different RDB products 5.

By way of example, in some instances, the system 10 can be configured to receive the RDB waste 15 that includes reduced density PLA. In such instances, the system 10 can recover the reduced density PLA, can generate a PLA resin, can dry the PLA resin, can extrude the PLA resin, and then can expand the PLA via one or more process described in the '590 patent and/or the '577 publication incorporated by reference herein. The expanded PLA can, in turn, be manufactured into any suitable reduced density PLA product.

In some instances, the recycled and subsequently expanded PLA can be used to form any suitable food service product such as those described above. In some instances, the expanded PLA used to form the food service products can be relatively high quality PLA. Similarly stated, the expanded PLA used to form the food service products can be substantially free of contaminants or can contain an amount of contaminants that is below a predetermined and/or desired threshold. In this context, contaminants can refer to any non-biopolymer and/or any non-PLA material that may otherwise alter and/or affect one or more desired characteristics of a pure PLA material.

While high quality PLA is desirable, in some instances, the system 10 can output and/or can generate PLA that has an amount of contaminants above the predetermined and/or desired threshold associated with food-grade products. In such instances, the non-food grade PLA material can be used to generate and/or form, for example, non-food grade products. For example, in some instances, non-food grade PLA can be used as a coating for one or more paper products. In such instances, the paper can be, for example, paper recovered and recycled by the system 10. In other instances, the non-food grade PLA can be used to generate and/or form one or more construction-grade products such as carpet fibers, plastic lumber, and/or the like. In still other instances, the non-food grade PLA can be used to generate and/or form one or more potting materials or growth mediums for plants.

As shown in FIG. 1, in some instances, the fermentation and/or digestion 19 can be configured to generate, co-generate, and/or output a biogas 25 in addition to or instead of the oligomers and/or monomers 21. For example, in some instances, the mixed waste feedstock 11 can be fermented as described above and a waste stream of the fermentation process (e.g., a portion of the mixed waste feedstock 11 not converted into the desired oligomers and/or monomers 21 during fermentation) can be further processed via one or more anaerobic digestion processes to produce the biogas 25. In some instances, the biogas 25 can be, for example, methane. In some embodiments, the methane can be produced and/or co-produced using the L. rhamnosus microorganism. In other embodiments, the methane can be produced and/or co-produced using one or more methanothropes that release methane in response to digesting organic material.

In some instances, the system 10 can be configured to convey the biogas 25 to an electric turbine 26, as shown in FIG. 1. In such instances, the electric turbine 26 can consume and/or otherwise use the methane to generate electric energy (e.g., electricity), which in turn, can be used to power equipment in the facility, stored in an energy storage facility, sold to one or more power companies and provided to the grid, and/or the like. Moreover, in some instances, waste heat 27 generated from the operation of the turbine 26 can be used to facilitate one or more of the pretreatment processes (e.g., Pretreatment 16, in FIG. 1), drying of the biopolymer (e.g., Dry Biopolymer 22, in FIG. 1), extruding of the biopolymer (e.g., Extruded Biopolymer 23, in FIG. 1), and/or expanding of the biopolymer (e.g., Expanded Biopolymer 24, in FIG. 1). In some instances, the system 10 can be configured to separate the biogas 25 (e.g., methane) from carbon dioxide (CO₂) that may be co-generated with, for example, methane. In such instances, the turbine 26 can be used to compress the CO₂, which in turn, can be used to expand the biopolymer and/or any other polymer (e.g., Expanded Biopolymer 24, in FIG. 1).

In some embodiments, at least a portion of the biogas 25 can be conveyed directly to one or more machines and/or pieces of equipment included in the pretreatment 16 and/or one or more machines and/or pieces of equipment configured to dry the biopolymer, as shown in FIG. 1. In such embodiments, the biogas 25 can be used, for example, as a fuel source for the one or more machines and/or pieces of equipment or can be used in one or more process performed by the machines or equipment.

While the system 10 is described above as generating and/or co-generating methane and/or any other biogas that can be used in one or more processes performed by the system 10 or converted into electric power via the turbine 26, in other instances, one or more organisms can be configured to generate a biopolymer from the methane and/or any other byproduct of the anaerobic digestion and/or fermentation. For example, in some instances, microorganisms such as, for example, methanothropes can be used to consume at least a portion the organic material and/or at least a portion of a methane byproduct and, in turn, can perform one or more processes and/or can generate one or more oligomers or monomers associated with a biopolymer such as, for example, PLA, PHA, PHB, and/or the like.

While the system 10 is described above as fermenting and/or digesting organic material having a specific gravity greater than a specific gravity threshold (also referred to as “high specific gravity material”), in some embodiments, the system 10 can be configured to treat the high specific gravity material via any suitable process. For example, in some instances, the system 10 can treat the high density material via hydrolysis before fermentation, during fermentation, or after fermentation. In some instances, performing hydrolysis on the high specific gravity material prior to fermentation may, for example, result in better separation of a biopolymer (e.g., PLA) from the mixed waste feedstock 11 and/or result in better or increased recovery of the biopolymer.

In some instances, for example, a higher density PLA (e.g., non-expanded PLA) and/or a reduced density PLA can be treated and/or further processed prior to being manufactured into a PLA product. For example, in some instances, the recycled PLA can be treated and/or processed with neat PLA (e.g., pure or virgin PLA), expanded PLA, one or more other biodegradable biopolymers or bioplastics, non-degraded or higher molecular weight polymers, pigments, chain extenders, crystal nucleators, fibers, fillers, and/or the like. Similarly, in some instances, the system 10 can further process and/or treat the recycled PLA by heating, compressing, compacting, chilling, electrolyzing, dehydrating, drying, etc., the recycled PLA.

While examples are provided above that describe the pretreatment 16, the separation 17 (e.g., float separation), the recycling 18, and the fermentation and/or digestion 19 being configured to separate PLA material, it should be understood that the system 10 can be configured to separate additional materials in one or more parallel and/or corresponding processes. For example, in some instances, the system 10 can be configured to separate cellulose and/or other similar organic materials during any one of the pretreatment 16, the separation 17 (e.g., float separation and/or any other suitable form of separation), the recycling 18, and/or the fermentation and/or digestion 19. In some instances, the separated and/or recovered cellulose can be processed into lactic acid, lactide, and/or ethyl lactate and/or can facilitate the processing of the recovered PLA into lactic acid and/or lactide oligomers and/or monomers. For example, in some instances, ethyl lactate can be used to facilitate and/or enhance fermentation and/or anaerobic digestion of organic material and/or PLA.

While various embodiments have been described above, it should be understood that they have been presented by way of example only, and not limitation. Where schematics and/or embodiments described above indicate certain components arranged in certain orientations or positions, the arrangement of components may be modified. While the embodiments have been particularly shown and described, it will be understood that various changes in form and details may be made. Although various embodiments have been described as having particular features and/or combinations of components, other embodiments are possible having a combination of any features and/or components from any of embodiments described herein.

The specific configurations of the various components can also be varied. For example, the size and specific shape of the various components can be different from the embodiments shown, while still providing the functions as described herein. More specifically, the size, shape, and/or arrangement of the various components can be specifically selected for a desired or intended usage such as, for example, separating specific and/or desired materials from a mixed waste feedstock. Thus, it should be understood that the size, shape, and/or arrangement of the embodiments and/or components thereof can be adapted for a given use unless the context explicitly states otherwise.

Where methods and/or events described above indicate certain events and/or procedures occurring in certain order, the ordering of certain events and/or procedures may be modified. Additionally, certain events and/or procedures may be performed concurrently in a parallel process when possible, as well as performed sequentially as described above.

1. A method for deriving value from a mixed waste feedstock, the method comprising:
receiving a mixed waste feedstock including at least a reduced density biopolymer material and an organic feedstock;
adding to the mixed waste feedstock at least one of a fluid or a material that releases liquids during degradation;
separating, via density separation, at least a portion of the reduced density biopolymer material from the mixed waste feedstock, the portion of the reduced density biopolymer material having a specific gravity less than a specific gravity threshold; and
recovering the portion of the reduced density biopolymer material separated from the mixed waste feedstock as a result of the separating.

2-3. (canceled)

4. The method of claim 1, wherein the portion of the reduced density biopolymer material is configured to float during the density separation.

5. The method of claim 4, wherein the recovering includes skimming the portion of the reduced density biopolymer material during the density separation.

6. The method of claim 1, wherein the reduced density biopolymer material has a layered structure with at least two distinct cell sizes.

7. The method of claim 1, wherein the reduced density biopolymer material has a durometer of at least 35 Shore A.

8. The method of claim 1, wherein the specific gravity threshold is 0.9.

9. The method of claim 1, further comprising:
exposing the mixed waste feedstock to autoclave conditions prior to the recovering of the portion of the reduced density biopolymer material.

10. The method of claim 1, wherein the mixed waste feedstock includes at least one of non-biopolymer material or metal material, the method further comprising:
separating at least a portion of the non-biopolymer material or the metal material from the mixed waste feedstock prior to the separating via density separation.

11. The method of claim 10, wherein the separating of the portion of the non-biopolymer material or the metal material from the mixed waste feedstock includes separating via source separation.

12. The method of claim 1, wherein the reduced density biopolymer material has a water-soluble barrier layer configured to be released during degradation.

13. (canceled)

14. The method of claim 1, wherein the separating via density separation results in a waste stream and the recovering includes recovering the portion of the reduced density biopolymer material separated from the waste stream, the method further comprising:
generating food packaging goods using the reduced density biopolymer material recovered via the recovering; and
generating non-food related goods using the waste stream.

15. The method of claim 1, wherein the mixed waste feedstock includes at least one non-biopolymer material, the at least one non-biopolymer material having a specific gravity greater than the specific gravity threshold.

16. The method of claim 15, wherein the specific gravity threshold is 0.9.

17. (canceled)

18. The method of claim 1, further comprising:
mixing air into the mixed waste feedstock during the separating via density separation.

19. The method of claim 18, further comprising:
mixing surfactants into the mixed waste feedstock during the separating via density separation, the surfactants configured to modify adsorption between particles and air bubbles resulting from the mixing of the air into the mixed waste feedstock to facilitate separation between hydrophilic material and hydrophobic material.

20-23. (canceled)

24. The method of claim 1, wherein the separating via density separation includes separating materials in the mixed waste feedstock having a specific gravity below the specific gravity threshold from materials in the mixed waste feedstock having a specific gravity above the specific gravity threshold, the materials with a specific gravity below the specific gravity threshold includes the portion of the reduced density biopolymer material and low density non-biopolymer materials, the method further comprising:
separating, via hydrolysis, the reduced density biopolymer material with a specific gravity below the specific gravity threshold from the non-biopolymer materials with a specific gravity below the specific gravity threshold.

25. The method of claim 1, wherein the separating via density separation includes separating materials in the mixed waste feedstock having a specific gravity below the specific gravity threshold from materials in the mixed waste feedstock having a specific gravity above the specific gravity threshold, the materials with a specific gravity below the specific gravity threshold includes the portion of the reduced density biopolymer material and low density non-biopolymer materials, the method further comprising:
separating, via hydrolysis, biopolymer materials with a specific gravity above the specific gravity threshold from other materials having a specific gravity above the specific gravity threshold.

26. (canceled)

39. The method of claim 1, further comprising:
separating cellulose from at least a portion of the mixed waste feedstock; and
processing the separated cellulose to generate at least one of lactic acid or ethyl lactate.

40-44. (canceled)

45. A method for deriving value from a mixed waste feedstock, the method comprising:
receiving a mixed waste feedstock composed of a reduced density biopolymer material and an organic feedstock;
adding to the mixed waste feedstock at least one of a fluid or a material that releases liquids during degradation; and
separating, via density separation, the reduced density biopolymer material from the mixed waste feedstock, the reduced density biopolymer material having a specific gravity below a specific gravity threshold;
recovering the reduced density biopolymer material separated from the mixed waste feedstock as a result of the separating; and
fermenting at least a portion of the mixed waste feedstock.

46. (canceled)

47. The method of claim 45, wherein the separating via density separation includes separating via float separation.

48. The method of claim 45, wherein the fermenting at least the portion of the mixed waste feedstock produces at least one of lactic acid or lactide.

49. The method of claim 45, wherein at least a portion of the reduced density biopolymer material having the specific gravity below the specific gravity threshold is operable as at least one of a nucleator or an initiator in a production of biopolymers.

50. The method of claim 45, further comprising:
separating cellulose from at least a portion of the mixed waste feedstock; and
processing the separated cellulose to generate at least one of lactic acid monomer, lactic acid oligomer, or ethyl lactate.

51. A method for deriving value from a mixed waste feedstock, the method comprising:
receiving a mixed waste feedstock composed of a reduced density biopolymer material and an organic feedstock;
adding to the mixed waste feedstock at least one of a fluid or a material that releases liquids during degradation;
separating, via density separation, the reduced density biopolymer material from the mixed waste feedstock, the reduced density biopolymer material having a specific gravity below a specific gravity threshold;
recovering the reduced density biopolymer material separated from the mixed waste feedstock as a result of the separating;
fermenting a portion of the mixed waste feedstock, the portion of the mixed waste feedstock including materials having a specific gravity greater than the specific gravity threshold;
generating biogas as a result of the fermenting; and
generating a biopolymer material via an organism.

52. (canceled)

53. The method of claim 51, further comprising:
providing at least a portion of the biogas to a turbine; and
receiving electric power generated by the turbine.

54-60. (canceled)
